(19) United States
(12) Patent Application Publication
(10) Pub. No.: US 2020/0306341 A1
(43) Pub. Date: Oct. 1, 2020

(54) TREATMENT OF EYE DISEASE

(71) Applicant: Oxford University Innovation Limited, Oxford Oxfordshire (GB)

(72) Inventors: Robert Maclaren, Oxford (GB); Alun R. Barnard, Oxford (GB); Samantha R. De Silva, Oxford (GB); Mark W. Hankins, Oxford (GB)

(21) Appl. No.: 16/738,616

(22) Filed: Jan. 9, 2020

Related U.S. Application Data
(63) Continuation of application No. 16/382,678, filed on Apr. 12, 2019, now abandoned.
Application No. 16/382,678 is a continuation of application No. 16/119,587, filed on Aug. 31, 2018, now abandoned.
Application No. 16/119,587 is a continuation of application No. 15/798,143, filed on Oct. 30, 2017, now abandoned.
Application No. 15/798,143 is a continuation of application No. 14/704,629, filed on May 5, 2015, now abandoned.

(51) Int. Cl. A61K 38/17; A61K 45/06; A61K 9/00; A61K 48/00; C07K 14/72

(52) U.S. Cl. CPC A61K 38/1709; A61K 45/06; A61K 38/177; A61K 9/0048; C12N 2750/14143; A61K 48/005; C07K 14/723; C12N 2710/10041; A61K 48/00

(57) ABSTRACT

A method of treating an eye disease comprising administering an adeno-associated virus (AAV) vector to a mammalian subject by subretinal injection, wherein the AAV vector comprises a nucleotide sequence encoding melanopsin operably linked to an expression control sequence to promote expression of melanopsin in cells of the eye of the subject.

### FIELD OF THE INVENTION

The present invention relates to the gene therapy of eye diseases. More specifically, the present invention relates to methods using adeno-associated virus (AAV) vectors in the treatment of eye diseases (e.g. retinitis pigmentosa, RP), wherein the AAV vectors enable delivery of melanopsin to the eye.

### BACKGROUND TO THE INVENTION

In the developed world, most causes of blindness occur due to the loss of photoreceptors in the eye.

Retinitis pigmentosa (RP) is one such condition that is commonly caused by the progressive degeneration of rod photoreceptor cells. During progression of the disease, the cone photoreceptor cells and retinal pigment epithelium (RPE) may also degenerate.

RP is a phenotypically linked group of inherited retinal dystrophies that leads to gradual reduction in vision. Early symptoms of RP include deterioration of night and peripheral vision. As the disease progresses, detailed, central and colour vision may also be affected. The age of onset of RP symptoms is variable, but typically between 10 and 30, and the rate of deterioration varies between individuals. RP affects approximately 1 in 3000-4000 people.

In late stages of RP, most if not all photoreceptor cells may degenerate, giving rise to complete blindness. However, the eye itself and the optic nerve remain relatively intact, which provides the potential to restore eyesight if the light-sensing cells can be replaced.

Previous studies using an electronic retinal implant have successfully restored some vision to blind subjects (Stingl K. et al. (2013) Invest. Ophthalmol. Vis. Sci. 54: 7658-65). However, the restored vision with electronic approaches may be limited and there may be long term biocompatibility challenges when electronic devices are permanently implanted into the eye.

An alternative approach taken in the prior art was to convert neurons that are not naturally light sensitive into photoreceptors by the introduction of the light sensitive protein melanopsin (Lin B. et al. (2008) Proc. Natl. Acad. Sci. USA 105: 16009-14). However, the study in Lin et al. was limited to targeting retinal ganglion cells for the incorporation of exogenous melanopsin, based on the knowledge that melanopsin is endogenously expressed in a small subset of that type of cell. For example, Lin et al. considered the need to harness signalling systems within the cells themselves as an important factor in determining a successful outcome. Although Lin et al. reports the restoration of simple visual function in a mouse model, the results are limited to crude visual resolution. Furthermore, the effects reported in Lin et al. may simply be an augmentation of the natural responses in the existing light-sensitive ganglion cells that already express melanopsin.

There is currently no approved therapy to improve vision following the onset of RP. Accordingly, there remains a significant need for treatments of RP, in particular to restore visual function in severely affected patients in the late stages of the disease.

### SUMMARY OF THE INVENTION

The present inventors have surprisingly shown that targeting melanopsin to retinal bipolar and horizontal cells, which are next in line after photoreceptor degeneration, provides sensitivity to light.

The present inventors have discovered that it is possible, for example, to target the retinal bipolar and horizontal cells by injecting the AAV vector under the retina (i.e. using subretinal injection). Furthermore, the present inventors have unexpectedly shown that an AAV vector harbouring a Y733F mutant AAV8 capsid protein improves the transduction of the target cells in the retina.

Using the inventor's subretinal injection approach, the AAV vector is not put in contact with naturally melanopsin expressing cells and so all resulting light sensitivity must come from the AAV vector treatment.

Targeting the bipolar and horizontal cells is advantageous because these cells more closely represent the map that would previously have been formed by the photoreceptors. Although ganglion cells could be made light-sensitive, the axons of the ganglion cells run horizontally across the retina to the optic nerve head and it is difficult to predict how vision would be useful without some form of electronic interface to reprogram the nerve signals.

Furthermore, gene therapy using intravitreal injections may be more prone to inflammation as the vector more easily escapes from the eye. It would also be possible to apply higher concentrations of the vector using the subretinal approach.

Targeting the bipolar and horizontal cells has the additional advantage of feeding the light-sensitive signals at the most upstream part of the normal visual pathway.

In summary, the present inventors have discovered an unexpected method of gene therapy that may be applied for the treatment of end stage RP, for example in situations where no functioning rod and cone photoreceptor cells remain in the affected eye.

Accordingly, in one aspect the present invention provides a method of treating an eye disease comprising administering an adeno-associated virus (AAV) vector to a mammalian subject by subretinal injection, wherein the AAV vector comprises a nucleotide sequence encoding melanopsin operably linked to an expression control sequence to promote expression of melanopsin in cells of the eye of the subject.

In one embodiment, the eye disease is a retinal dystrophy. In another embodiment, the eye disease is selected from retinitis pigmentosa, macular degeneration, age-related macular degeneration or diabetic retinopathy. Preferably, the eye disease is retinitis pigmentosa.

In one embodiment, the eye to be treated has less than 50%, 40%, 30%, 20%, 10%, 5%, 4%, 3%, 2% or 1% of the functional rod and/or cone photoreceptor cells that were present prior to the onset of the disease.

In one embodiment, the eye to be treated substantially lacks rod and/or cone photoreceptor cells. The present invention advantageously may restore or improve vision in an eye in which substantially all rod and/or cone photoreceptor cells have degenerated due to disease. For example, the subject to be treated may be blind in the eye to be treated prior to administration of the AAV vector of the invention.

Preferably, the AAV vector is in the form of an AAV vector particle.

The AAV vector of the invention may comprise a genome and/or capsid of any serotype, provided that the vector is capable of mediating the expression of melanopsin in cells of the eye.

In one embodiment, the AAV vector comprises an AAV capsid protein of serotype 1, 2, 3, 4, 5, 6, 7, 8, 9, 10, or 11. In another embodiment, the AAV vector comprises an AAV capsid protein of serotype 2, 5 or 8. Preferably, the AAV vector comprises an AAV capsid protein of serotype 8. More preferably, the AAV vector is in the form of an AAV particle comprising an AAV8 Y733F mutant capsid.

In one embodiment, the AAV vector comprises an AAV serotype 1, 2, 3, 4, 5, 6, 7, 8, 9, 10, or 11 genome. In another embodiment, the AAV vector comprises an AAV serotype 2, 4, 5 or 8 genome. Preferably, the AAV vector comprises an AAV serotype 2 genome.

In one embodiment, the AAV vector comprises AAV2 ITRs.

In one embodiment, the AAV vector particle comprises an AAV2 genome and AAV2 capsid proteins (AAV2/2); an AAV2 genome and AAV5 capsid proteins (AAV2/5); or an AAV2 genome and AAV8 capsid proteins (AAV2/8). Preferably, the AAV vector particle comprises an AAV2 genome and AAV8 capsid proteins (AAV2/8), particularly preferably AAV8 Y733F mutant capsid proteins.

The AAV vector particle of the invention may be a chimeric, shuffled or capsid-modified derivative of one or more naturally occurring AAVs. In particular, the AAV vector particle may comprise capsid protein sequences from different serotypes, clades, clones or isolates of AAV within the same vector (i.e. pseudotyping). Thus, in one embodiment the AAV vector is in the form of a pseudotyped AAV vector particle.

Preferably, the melanopsin is expressed in bipolar and/or horizontal cells.

In one embodiment, the melanopsin is human melanopsin.

In another embodiment, the melanopsin-encoding nucleotide sequence is selected from the group consisting of:
(a) a nucleotide sequence having at least 70% identity to SEQ ID NO: 1 or 2; and
(b) a nucleotide sequence encoding an amino acid sequence having at least 70% identity to SEQ ID NO: 3 or 4,
wherein the protein encoded by the nucleotide sequence substantially retains the natural function of the protein represented by SEQ ID NO: 3 or 4.

In one embodiment, the expression control sequence comprises a CBA promoter.

In one embodiment, the subretinal injection comprises the steps:
(a) administering a solution to the subject by subretinal injection in an amount effective to at least partially detach the retina to form a subretinal bleb, wherein the solution does not comprise the AAV vector; and
(b) administering a medicament composition by subretinal injection into the bleb formed by step (a), wherein the medicament comprises the AAV vector.

The AAV vector may, for example, be in a suspension at a concentration of about 1-2×10¹¹,1-2×10¹² or 1-2×10¹³genome particles (gp) per mL. Thus a dose of AAV vector of about 2×10¹⁰ gp may, for example, be administered by injecting about a 10 μL dose of AAV vector at a concentration of about 2×10¹² gp per mL. The skilled person is readily able to adjust the dose, volume and concentration of the AAV vector as necessary.

The volume of the AAV vector administered may be, for example, about 1-500 μL, for example about 10-500, 50-500, 100-500, 200-500, 300-500, 400-500, 50-250, 100-250, 200-250, 50-150, 1-100 or 1-10 μL. The volume may be, for example, about 1, 2, 5, 10, 50, 100, 150, 200, 250, 300, 350, 400, 450 or 500 μL. Preferably, the volume of the AAV vector composition injected is about 100 μL.

In one embodiment, the AAV vector is administered at a dosage of at least 2×10⁹, 2×10¹⁰, 2×10¹¹ or 2×10¹² gp per eye. In another embodiment, the AAV vector is administered at a dosage of about 1-2×10⁹, 1-2×10¹, 1-2×10¹¹ or 1-2×10¹² gp per eye. Preferably, the AAV vector is administered at a dosage of about 2×10¹¹ gp per eye by subretinal injection.

In another aspect, the present invention provides a method of improving or restoring vision comprising administering an adeno-associated virus (AAV) vector to a mammalian subject by subretinal injection, wherein the AAV vector comprises a nucleotide sequence encoding melanopsin operably linked to an expression control sequence to promote expression of melanopsin in cells of the eye of the subject.

The nature of the subject's disease or condition, the AAV vector and mode (e.g. method and dosage) of administration may be as described herein.

### DESCRIPTION OF THE DRAWINGS

FIGS. 1A-D: Long term expression of human melanopsin in the degenerate retina following subretinal delivery using an adeno-associated virus (AAV) vector.

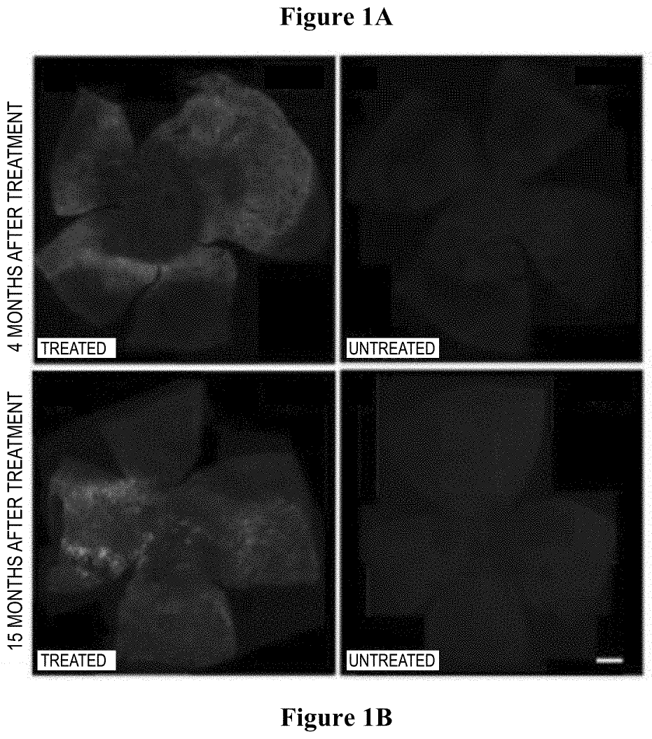

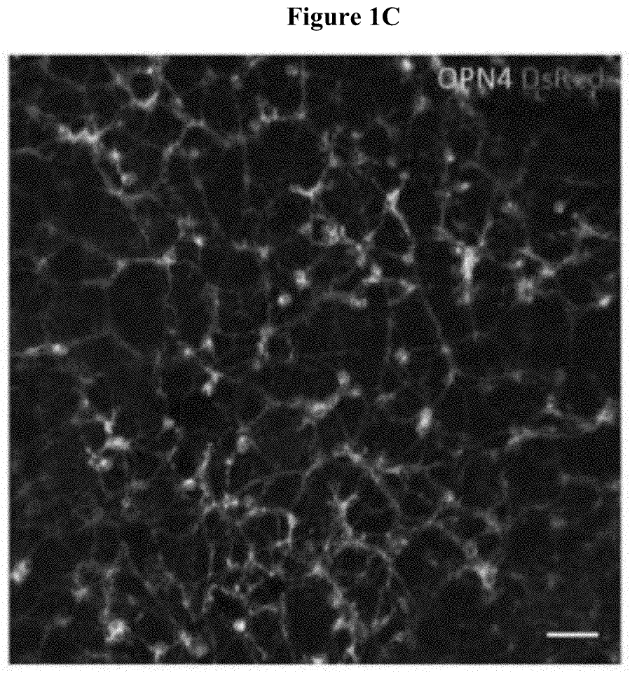

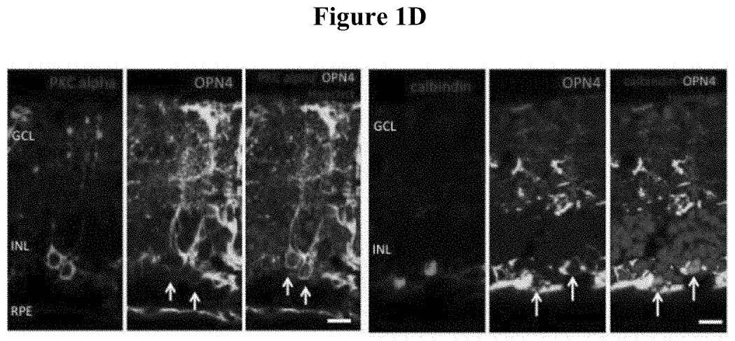

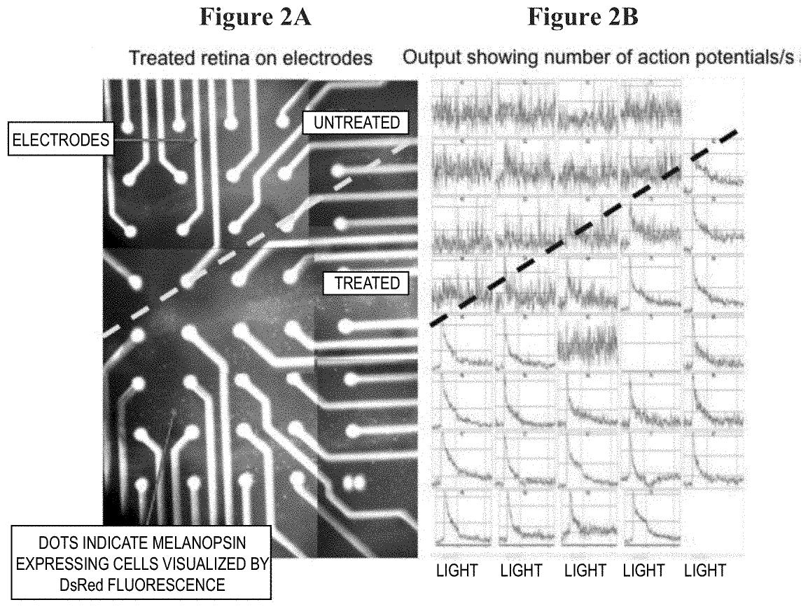

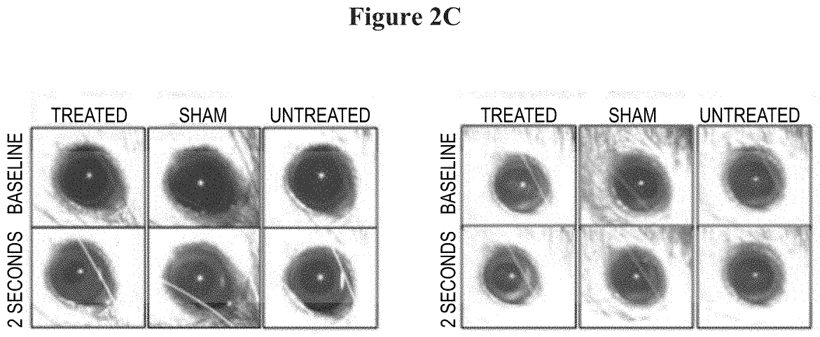

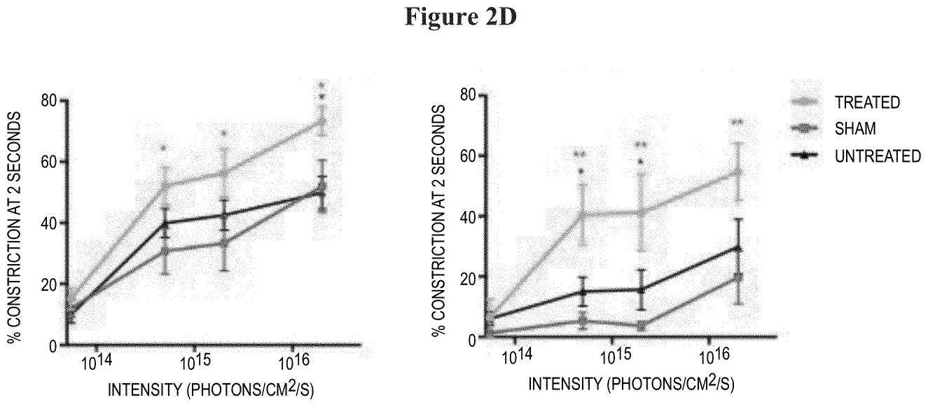

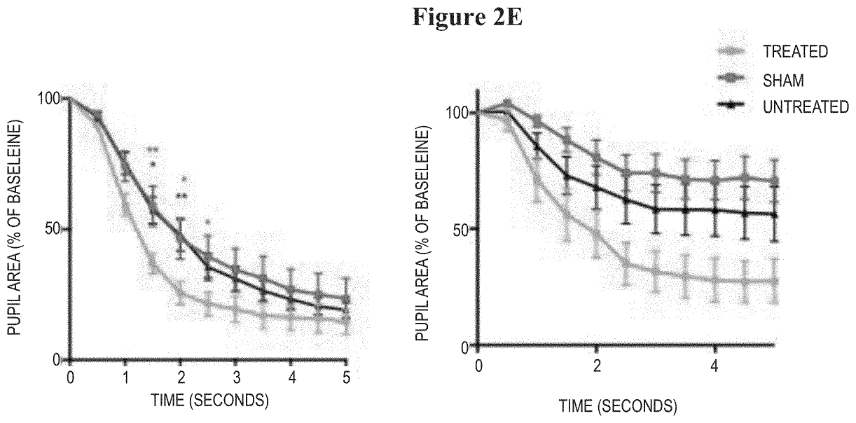

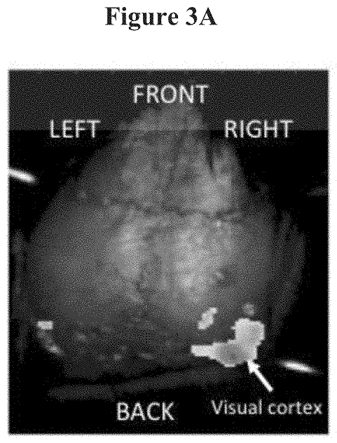

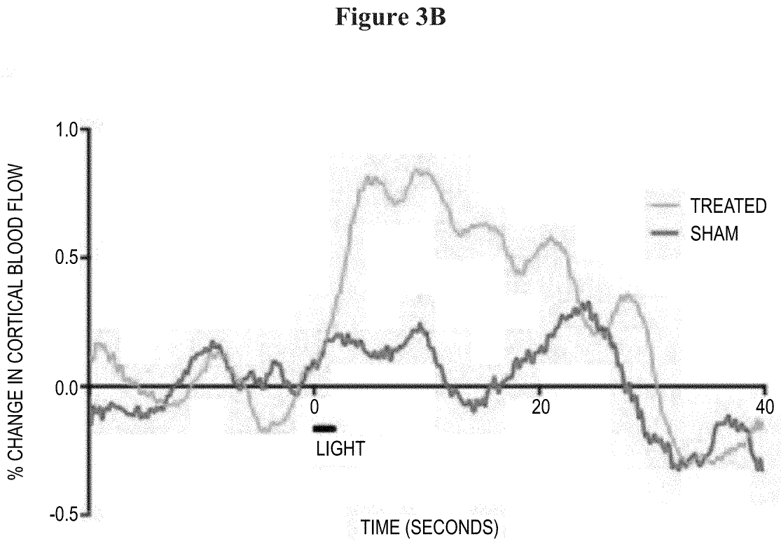

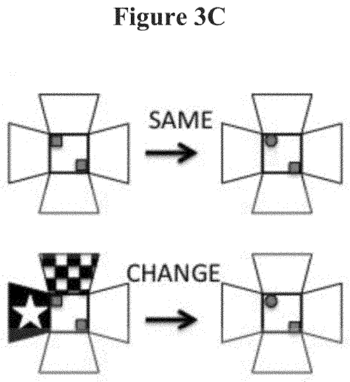

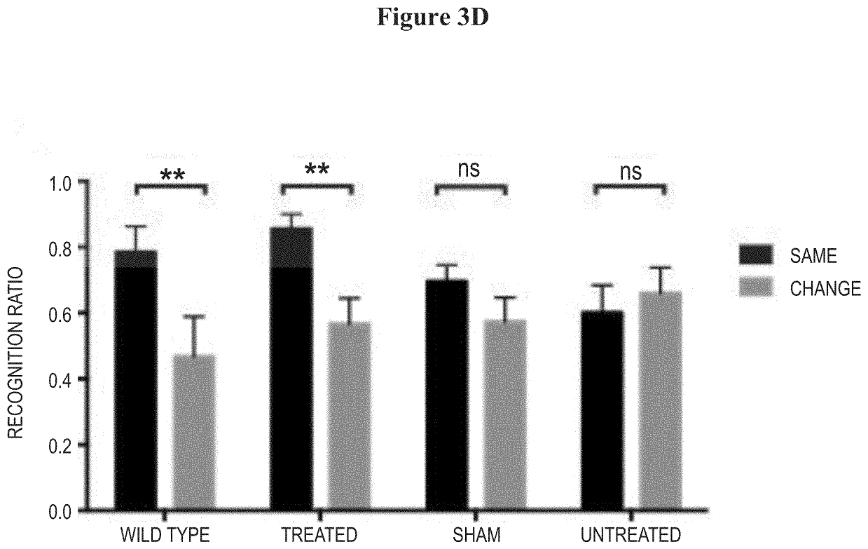

Widespread transduction of the retina of the rd1 mouse (which has a rapid photoreceptor degeneration) immune-stained in green for human melanopsin—4 months (FIG. 1A, left image) and 15 months (FIG. 1B, left image) after gene therapy. Images of control retinae are shown for comparison showing absence of human melanopsin at 4 months (FIG. 1A, right image) and 15 months (FIG. 1B, right image). The red label (DsRed) in FIG. 1C identifies transduced bipolar and horizontal cells on the subretinal surface. Appropriate membrane localisation of human melanopsin is also evident demonstrating the network of transduced cells. Immunostaining for PKC alpha identifying bipolar cells and calbindin identifying horizontal cells (series of six panels in FIG. 1D) illustrates transduction of these cell types in the degenerate retina using the melanopsin vector.

FIGS. 2A-C: Human melanopsin expressed by an AAV vector in the degenerate retina is functional and able to drive light responses.

Multi-electrode array (MEA) recordings from an ex vivo rd1 mouse retina six months after treatment with melanopsin gene therapy. The lower half of the field below the yellow dotted line shows fluorescent dots (DsRed) which confirm the area of the retina transduced with the melanopsin expressing vector (FIG. 2A). Corresponding electrode recordings are shown in panel (FIG. 2B). The MEA recordings taken from retinal ganglion cells show action potentials corresponding to the regions of transduced retina below. These specific ganglion cell outputs could convey a retinotopic map of a visual image to the central visual centres.

Pupil responses in blind mice two months (FIG. 2C, left panel) and twelve months (FIG. 2C, right panel) after transduction of the residual outer retina with 2×10⁹ genome particles of AAV vector with a Y733F mutant AAV8 capsid protein. The irradiance response curves (FIG. 2D) shows the mean and standard error of three cohorts of mice with significantly enhanced pupil constriction seen in the AAV vector treated group compared with sham-injected controls (*) and untreated retina (*). The response kinetics of the pupil constriction curve (FIG. 2E) following a stimulus of 2×10¹⁶photons/cm²/s shows a slight delay (approximately 200 ms). The effects seen in the treated group at 2 months (FIG. 2D-2E, left panels) are sustained at 12 months (FIG. 2D-2E, right panels).

FIGS. 3A-D: Human melanopsin expression in the degenerate retina may restore image-forming vision.

The technique of laser speckle cortical imaging was used to assess blood flow changes in the visual cortex following a 2 second 480 nm light stimulus. A photograph of exposed skull is shown in FIG. 3A, indicating orientation and the location of the visual cortex. Activation in response to visual stimulation in a treated mouse is overlaid, with a large responsecorresponding to the right visual cortex, and a small response corresponding to the left visualcortex. Blood flow changes in the visual cortex were measured in treated (n=6) and sham-injected (n=5) mice. Mean responses in both groups are shown in FIG. 3B.

Visual environment recognition test in mice treated with human melanopsin compared to controls at 12 months after injection. Schematic FIG. 3C illustrates the test where each animal has two trials where the visual environment is constant (same) or there is a change in visual environment during the task from a black and white patterned box to a plain white box (change). Measure of behaviour in FIG. 3D with and without change in environment for each group demonstrating a significant difference in behaviour in wild type and treated mice when the visual environment is changed (** p<0.01, 2 way ANOVA with Tukey post hoc test).

### DETAILED DESCRIPTION OF THE INVENTION

Various preferred features and embodiments of the present invention will now be described by way of non-limiting examples.

The practice of the present invention will employ, unless otherwise indicated, conventional techniques of chemistry, biochemistry, molecular biology, microbiology and immunology, which are within the capabilities of a person of ordinary skill in the art. Such techniques are explained in the literature. See, for example, Sambrook, J., Fritsch, E. F. and Maniatis, T. (1989) Molecular Cloning: A Laboratory Manual, 2nd Edition, Cold Spring Harbor Laboratory Press; Ausubel, F. M. et al. (1995 and periodic supplements) Current Protocols in Molecular Biology, Ch. 9, 13 and 16, John Wiley & Sons; Roe, B., Crabtree, J. and Kahn, A. (1996) DNA Isolation and Sequencing: Essential Techniques, John Wiley & Sons; Polak, J. M. and McGee, J. O'D. (1990) In Situ Hybridization: Principles and Practice, Oxford University Press; Gait, M. J. (1984) Oligonucleotide Synthesis: A Practical Approach, IRL Press; and Lilley, D. M. and Dahlberg, J. E. (1992) Methods in Enzymology: DNA Structures Part A: Synthesis and Physical Analysis of DNA, Academic Press. Each of these general texts is herein incorporated by reference.

In one aspect the present invention provides a method of treating an eye disease comprising administering an adeno-associated virus (AAV) vector to a mammalian subject by subretinal injection, wherein the AAV vector comprises a nucleotide sequence encoding melanopsin operably linked to an expression control sequence to promote expression of melanopsin in cells of the eye of the subject.

### Retinitis Pigmentosa (RP)

Retinitis pigmentosa (RP) is a phenotypically linked group of inherited retinal dystrophies which is commonly caused by the progressive degeneration of rod photoreceptor cells. The cone photoreceptor cells and retinal pigment epithelium (RPE) may also degenerate during progression of the disease. In late stage RP, most if not all rod and cone photoreceptors may be lost, which may result in blindness.

RP is characterised in clinical appearance by changes in the pigment of the retina, which may be accompanied by arteriolar attenuation and optic nerve atrophy. Changes in the retina may result from dispersion and aggregation of the retinal pigment. This may give rise to an appearance ranging from granular or mottled to distinctive focal aggregates resembling bone spicules. Black or dark brown star-shaped concentrations of pigment may appear. Furthermore, pigmentation limited to one quadrant of the retina, abnormalities which appear to be radiating out from the disc and changes associated with severe vasculopathy may be observed.

The method of treatment of RP described herein may, for example, be used to improve or restore visual function in subjects. Preferably, the treatment improves or restores visual function to an eye substantially lacking rod and cone photoreceptor cells.

Numbers of rods and cones can be estimated in the clinic by the skilled person using techniques such as adaptive optics, autofluorescence and optical coherence tomography (OCT) scans.

Visualisation of the appearance of a retina and assessment of visual function may also be readily carried out by the skilled person. Applicable visual function tests include, for example, best corrected visual acuity, visual field testing, microperimetry, colour vision, dark adaptometry, electroretinography and cone flicker fusion tests.

To confirm that a subject's optic nerve and central visual relays are intact, despite loss of photoreceptors, an electrically evoked phosphene (EEP) test may be carried out. This test confirms that potential subjects can experience light signals when the retina is stimulated with a current applied across a contact lens.

Preferably subjects to be treated will not exhibit significant loss of the retinal pigment epithelium (RPE), because melanopsin is dependent on the 11 cis-retinal, which is recycled from the all trans-retinal in the RPE.

### Structure of the Eye

The method of the present invention requires the delivery of a medicament to a mammalian, preferably human eye.

The person skilled in the treatment of diseases of the eye will have a detailed and thorough understanding of the structure of the eye. However, the following structures of particular relevance to the present invention are described.

### Retina

The retina is the multi-layered membrane which lines the inner posterior chamber of the eye and senses an image of the visual world which is communicated to the brain via the optic nerve. In order from the inside to the outside of the eye, the retina comprises the layers of the neurosensory retina and retinal pigment epithelium, with the choroid lying outside the retinal pigment epithelium.

### Neurosensory Retina and Photoreceptor Cells

The neurosensory retina harbours the photoreceptor cells that directly sense light. It comprises the following layers: internal limiting membrane (ILM); nerve fibre layer; ganglion cell layer; inner plexiform layer; inner nuclear layer; outer plexiform layer; outer nuclear layer (nuclei of the photoreceptors); external limiting membrane (ELM); and photoreceptors (inner and outer segments) of the rods and cones.

The skilled person will have a detailed understanding of photoreceptor cells. Briefly, photoreceptor cells are specialised neurons located in the retina that convert light into biological signals. Photoreceptor cells comprise rod and cone cells, which are distributed differently across the retina.

Rod cells are distributed mainly across the outer parts of the retina. They are highly sensitive and provide for vision at low light levels.

Cone cells are found across the retina, but are particular highly concentrated in the fovea, a pit in the neurosensory retina that is responsible for central high resolution vision. Cone cells are less sensitive than rod cells.

In addition to the rod and cone photoreceptor cells, the retina comprises a number of other neurons including bipolar cells, horizontal cells, amacrine cells and ganglion cells. The skilled person will have a detailed understanding of these cell types.

Briefly, bipolar cells are located in the retina adjacent to the photoreceptors. They transmit signals from the rod and cone cells to amacrine and/or ganglion cells. Bipolar cells may also synapse with horizontal cells.

Horizontal cells are located in the inner nuclear layer and are involved in signal processing and feedback to both photoreceptor and bipolar cells. They help integrate and regulate input from multiple photoreceptor cells.

Amacrine cells are located in the inner plexiform layer and act as a bridge between the photoreceptor pathway and ganglion cells.

Ganglion cells are located in the inner-most region of the retina. They transmit signals originating from the photoreceptor cells through the optic fibre layer to the origin of the optic nerve.

### Retinal Pigment Epithelium

The retinal pigment epithelium (RPE) is a pigmented layer of cells located immediately to the outside of the neurosensory retina. The RPE performs a number of functions, including transport of nutrients and other substances to the photoreceptor cells, and absorption of scattered light to improve vision.

### Choroid

The choroid is the vascular layer situated between the RPE and the outer sclera of the eye. The vasculature of the choroid enables provision of oxygen and nutrients to the retina.

### Melanopsin

Melanopsin is a membrane-bound light-sensitive protein similar to the rhodopsin present in photoreceptors. Melanopsin differs from rhodopsin in that it activates a second messenger system which is present in all neurons and not just rod and cone photoreceptors. Melanopsin is naturally located in a small subset of retinal ganglion cells. It contributes to pupil light response, and also functions to regulate circadian rhythm and the recognition of light-dark cycles.

Melanopsin may be encoded by the OPN4 gene. The melanopsin protein may also sometimes be referred to as OPN4.

In one embodiment of the present invention, the melanopsin is human melanopsin.

In one embodiment, the nucleotide sequence encoding melanopsin is the sequence deposited under NCBI Accession No. NM_033282.3.

In another embodiment, the nucleotide sequence encoding melanopsin is:

(SEQ ID NO: 1)
ATGAACCCTCCTTCGGGGCCAAGAGTCCCGCCCAGCCCAACCCAAGAGCC
CAGCTGCATGGCCACCCCAGCACCACCCAGCTGGTGGGACAGCTCCCAGA
GCAGCATCTCCAGCCTGGGCCGGCTTCCATCCATCAGTCCCACAGCACCT
GGGACTTGGGCTGCTGCCTGGGTCCCCCTCCCCACGGTTGATGTTCCAGA
CCATGCCCACTATACCCTGGGCACAGTGATCTTGCTGGTGGGACTCACGG
GGATGCTGGGCAACCTGACGGTCATCTATACCTTCTGCAGGAGCAGAAGC
CTCCGGACACCTGCCAACATGTTCATTATCAACCTCGCGGTCAGCGACTT
CCTCATGTCCTTCACCCAGGCCCCTGTCTTCTTCACCAGTAGCCTCTATA
AGCAGTGGCTCTTTGGGGAGACAGGCTGCGAGTTCTATGCCTTCTGTGGA
GCTCTCTTTGGCATTTCCTCCATGATCACCCTGACGGCCATCGCCCTGGA
CCGCTACCTGGTAATCACACGCCCGCTGGCCACCTTTGGTGTGGCGTCCA
AGAGGCGTGCGGCATTTGTCCTGCTGGGCGTTTGGCTCTATGCCCTGGCC
TGGAGTCTGCCACCCTTCTTCGGCTGGAGCGCCTACGTGCCCGAGGGGTT
GCTGACATCCTGCTCCTGGGACTACATGAGCTTCACGCCGGCCGTGCGTG
CCTACACCATGCTTCTCTGCTGCTTCGTGTTCTTCCTCCCTCTGCTTATC
ATCATCTACTGCTACATCTTCATCTTCAGGGCCATCCGGGAGACAGGACG
GGCTCTCCAGACCTTCGGGGCCTGCAAGGGCAATGGCGAGTCCCTGTGGC
AGCGGCAGCGGCTGCAGAGCGAGTGCAAGATGGCCAAGATCATGCTGCTG
GTCATCCTCCTCTTCGTGCTCTCCTGGGCTCCCTATTCCGCTGTGGCCCT
GGTGGCCTTTGCTGGGTACGCACACGTCCTGACACCCTACATGAGCTCGG
TGCCAGCCGTCATCGCCAAGGCCTCTGCAATCCACAACCCCATCATTTAC
GCCATCACCCACCCCAAGTACAGGGTGGCCATTGCCCAGCACCTGCCCTG
CCTGGGGGTGCTGCTGGGTGTATCACGCCGGCACAGTCGCCCCTACCCCA
GCTACCGCTCCACCCACCGCTCCACGCTGACCAGCCACACCTCCAACCTC
AGCTGGATCTCCATACGGAGGCGCCAGGAGTCCCTGGGCTCGGAGAGTGA
GGTGGGCTGGACACACATGGAGGCAGCAGCTGTGTGGGGAGCTGCCCAGC
AAGCAAATGGGCGGTCCCTCTACGGTCAGGGTCTGGAGGACTTGGAAGCC
AAGGCACCCCCCAGACCCCAGGGACACGAAGCAGAGACTCCAGGGAAGAC
CAAGGGGCTGATCCCCAGCCAGGACCCCAGGATGTAG

In one embodiment, the nucleotide sequence encoding melanopsin is the sequence deposited under NCBI Accession No. NM_001030015.2.

In another embodiment, the nucleotide sequence encoding melanopsin is:

(SEQ ID NO: 2)
ATGAACCCTCCTTCGGGGCCAAGAGTCCCGCCCAGCCCAACCCAAGAGCC
CAGCTGCATGGCCACCCCAGCACCACCCAGCTGGTGGGACAGCTCCCAGA
GCAGCATCTCCAGCCTGGGCCGGCTTCCATCCATCAGTCCCACAGCACCT
GGGACTTGGGCTGCTGCCTGGGTCCCCCTCCCCACGGTTGATGTTCCAGA
CCATGCCCACTATACCCTGGGCACAGTGATCTTGCTGGTGGGACTCACGG
GGATGCTGGGCAACCTGACGGTCATCTATACCTTCTGCAGAGCTGTGCTT
CGTGGAGTCACTGTGATGATGCAGAGCAGAAGCCTCCGGACACCTGCCAA
CATGTTCATTATCAACCTCGCGGTCAGCGACTTCCTCATGTCCTTCACCC
AGGCCCCTGTCTTCTTCACCAGTAGCCTCTATAAGCAGTGGCTCTTTGGG
GAGACAGGCTGCGAGTTCTATGCCTTCTGTGGAGCTCTCTTTGGCATTTC
CTCCATGATCACCCTGACGGCCATCGCCCTGGACCGCTACCTGGTAATCA
CACGCCCGCTGGCCACCTTTGGTGTGGCGTCCAAGAGGCGTGCGGCATTT
GTCCTGCTGGGCGTTTGGCTCTATGCCCTGGCCTGGAGTCTGCCACCCTT
CTTCGGCTGGAGCGCCTACGTGCCCGAGGGGTTGCTGACATCCTGCTCCT
GGGACTACATGAGCTTCACGCCGGCCGTGCGTGCCTACACCATGCTTCTC
TGCTGCTTCGTGTTCTTCCTCCCTCTGCTTATCATCATCTACTGCTACAT
CTTCATCTTCAGGGCCATCCGGGAGACAGGACGGGCTCTCCAGACCTTCG
GGGCCTGCAAGGGCAATGGCGAGTCCCTGTGGCAGCGGCAGCGGCTGCAG
AGCGAGTGCAAGATGGCCAAGATCATGCTGCTGGTCATCCTCCTCTTCGT
GCTCTCCTGGGCTCCCTATTCCGCTGTGGCCCTGGTGGCCTTTGCTGGGT
ACGCACACGTCCTGACACCCTACATGAGCTCGGTGCCAGCCGTCATCGCC
AAGGCCTCTGCAATCCACAACCCCATCATTTACGCCATCACCCACCCCAA
GTACAGGGTGGCCATTGCCCAGCACCTGCCCTGCCTGGGGGTGCTGCTGG
GTGTATCACGCCGGCACAGTCGCCCCTACCCCAGCTACCGCTCCACCCAC
CGCTCCACGCTGACCAGCCACACCTCCAACCTCAGCTGGATCTCAATACG
GAGGCGCCAGGAGTCCCTGGGCTCGGAGAGTGAGGTGGGCTGGACACACA
TGGAGGCAGCAGCTGTGTGGGGAGCTGCCCAGCAAGCAAATGGGCGGTCC
CTCTACGGTCAGGGTCTGGAGGACTTGGAAGCCAAGGCACCCCCCAGACC
CCAGGGACACGAAGCAGAGACTCCAGGGAAGACCAAGGGGCTGATCCCCA
GCCAGGACCCCAGGATGTAG

In one embodiment, the amino acid sequence of melanopsin is the sequence deposited under NCBI Accession No. NP_150598.1.

In another embodiment, the amino acid sequence of melanopsin is:

(SEQ ID NO: 3)
MNPPSGPRVPPSPTQEPSCMATPAPPSWWDSSQSSISSLGRLPSISPTAP
GTWAAAWVPLPTVDVPDHAHYTLGTVILLVGLTGMLGNLTVIYTFCRSRS
LRTPANMFIINLAVSDFLMSFTQAPVFFTSSLYKQWLFGETGCEFYAFCG
ALFGISSMITLTAIALDRYLVITRPLATFGVASKRRAAFVLLGVWLYALA
WSLPPFFGWSAYVPEGLLTSCSWDYMSFTPAVRAYTMLLCCFVFFLPLLI
IIYCYIFIFRAIRETGRALQTFGACKGNGESLWQRQRLQSECKMAKIMLL
VILLFVLSWAPYSAVALVAFAGYAHVLTPYMSSVPAVIAKASAIHNPIIY
AITHPKYRVAIAQHLPCLGVLLGVSRRHSRPYPSYRSTHRSTLTSHTSNL
SWISIRRRQESLGSESEVGWTHMEAAAVWGAAQQANGRSLYGQGLEDLEA
KAPPRPQGHEAETPGKTKGLIPSQDPRM

In one embodiment, the amino acid sequence of melanop sin is the sequence deposited under NCBI Accession No. NP_001025186.1.

In another embodiment, the amino acid sequence of melanopsin is:

(SEQ ID NO: 4)
MNPPSGPRVPPSPTQEPSCMATPAPPSWWDSSQSSISSLGRLPSISPTAP
GTWAAAWVPLPTVDVPDHAHYTLGTVILLVGLTGMLVNLTVIYTFCRAVL
RGVTVMMQSRSLRTPANMFIINLAVSDFLMSFTQAPVFFTSSLYKQWLFG
ETGCEFYAFCGALFGISSMITLTAIALDRYLVITRPLATFGVASKRRAAF
VLLGVWLYALAWSLPPFFGWSAYVPEGLLTSCSWDYMSFTPAVRAYTMLL
CCFVFFLPLLIIIYCYIFIFRAIRETGRALQTFGACKGNGESLWQRQRLQ
SECKMAKIMLLVILLFVLSWAPYSAVALVAFAGYAHVLTPYMSSVPAVIA
KASAIHNPIIYAITHPKYRVAIAQHLPCLGVLLGVSRRHSRPYPSYRSTH
RSTLTSHTSNLSWISIRRRQESLGSESEVGWTHMEAAAVWGAAQQANGRS
LYGQGLEDLEAKAPPRPQGHEAETPGKTKGLIPSQDPRM

The nucleotide sequence encoding melanopsin of the present invention may, for example, comprise a nucleotide sequence that has at least 70%, 80%, 90%, 95%, 96%, 97%, 98% 99% or 100% identity to SEQ ID NO: 1 or 2, wherein the protein encoded by the nucleotide sequence substantially retains the natural function of the protein represented by SEQ ID NO: 3 or 4.

The nucleotide sequence encoding melanopsin of the present invention may, for example, encode an amino acid sequence that has at least 70%, 80%, 90%, 95%, 96%, 97%, 98% 99% or 100% identity to SEQ ID NO: 3 or 4, wherein the amino acid sequence substantially retains the natural function of the protein represented by SEQ ID NO: 3 or 4.

Preferably, the nucleotide sequence of the present invention encodes a protein which assists in providing similar or higher light sensitivity or visual function when expressed in retinal bipolar and/or horizontal cells in a subject suffering from RP compared to the protein of SEQ ID NO: 3 or 4.

### Vectors

A vector is a tool that allows or facilitates the transfer of an entity from one environment to another.

The vectors used in the present invention may include a promoter for the expression of a polynucleotide and optionally a regulator of the promoter.

### Adeno-Associated Virus (AAV) Vectors

The vector of the present invention is an adeno-associated virus (AAV) vector. Preferably, the AAV vector is in the form of an AAV vector particle.

Methods of preparing and modifying viral vectors and viral vector particles, such as those derived from AAV, are well known in the art and can be readily adapted by the skilled person to the required purpose.

The AAV vector may comprise an AAV genome or a derivative thereof.

An AAV genome is a polynucleotide sequence which encodes functions needed for production of an AAV particle. These functions include those operating in the replication and packaging cycle of AAV in a host cell, including encapsidation of the AAV genome into an AAV particle. Naturally occurring AAVs are replication-deficient and rely on the provision of helper functions in trans for completion of a replication and packaging cycle. Accordingly, the AAV genome of the vector of the invention is typically replication-deficient.

The AAV genome may be in single-stranded form, either positive or negative-sense, or alternatively in double-stranded form. The use of a double-stranded form allows bypass of the DNA replication step in the target cell and so can accelerate transgene expression.

The AAV genome may be from any naturally derived serotype, isolate or clade of AAV. Thus, the AAV genome may be the full genome of a naturally occurring AAV. As is known to the skilled person, AAVs occurring in nature may be classified according to various biological systems.

Commonly, AAVs are referred to in terms of their serotype. A serotype corresponds to a variant subspecies of AAV which, owing to its profile of expression of capsid surface antigens, has a distinctive reactivity which can be used to distinguish it from other variant subspecies. Typically, a virus having a particular AAV serotype does not efficiently cross-react with neutralising antibodies specific for any other AAV serotype.

AAV serotypes include AAV1, AAV2, AAV3, AAV4, AAV5, AAV6, AAV7, AAV8, AAV9, AAV10 and AAV11, and also recombinant serotypes, such as Rec2 and Rec3, recently identified from primate brain. Any of these AAV serotypes may be used in the present invention. Thus, in one embodiment of the present invention, the AAV vector particle is an AAV1, AAV2, AAV3, AAV4, AAV5, AAV6, AAV7, AAV8, AAV9, AAV10, AAV11, Rec2 or Rec3 AAV vector particle.

Preferred serotypes for use in the invention are AAV2, AAV4, AAV5 and AAV8.

Reviews of AAV serotypes may be found in Choi et al. (2005) Curr. Gene Ther. 5: 299-310 and Wu et al. (2006) Molecular Therapy 14: 316-27. The sequences of AAV genomes or of elements of AAV genomes including ITR sequences, rep or cap genes for use in the invention may be derived from the following accession numbers for AAV whole genome sequences: Adeno-associated virus 1 NC_002077, AF063497; Adeno-associated virus 2 NC_001401; Adeno-associated virus 3 NC_001729; Adeno-associated virus 3B NC_001863; Adeno-associated virus 4 NC_001829; Adeno-associated virus 5 Y18065, AF085716; Adeno-associated virus 6 NC_001862; Avian AAV ATCC VR-865 AY186198, AY629583, NC_004828; Avian AAV strain DA-1 NC_006263, AY629583; Bovine AAV NC_005889, AY388617.

AAV may also be referred to in terms of clades or clones. This refers to the phylogenetic relationship of naturally derived AAVs, and typically to a phylogenetic group of AAVs which can be traced back to a common ancestor, and includes all descendants thereof. Additionally, AAVs may be referred to in terms of a specific isolate, i.e. a genetic isolate of a specific AAV found in nature. The term genetic isolate describes a population of AAVs which has undergone limited genetic mixing with other naturally occurring AAVs, thereby defining a recognisably distinct population at a genetic level.

The skilled person can select an appropriate serotype, clade, clone or isolate of AAV for use in the present invention on the basis of their common general knowledge. For instance, the AAV5 capsid has been shown to transduce primate cone photoreceptors efficiently as evidenced by the successful correction of an inherited colour vision defect (Mancuso et al. (2009) Nature 461: 784-7).

The AAV serotype determines the tissue specificity of infection (or tropism) of an AAV virus. Accordingly, preferred AAV serotypes for use in AAVs administered to patients in accordance with the invention are those which have natural tropism for or a high efficiency of infection of target cells within the eye. In one embodiment, AAV serotypes for use in the present invention are those which infect bipolar and/or horizontal cells.

Typically, the AAV genome of a naturally derived serotype, isolate or clade of AAV comprises at least one inverted terminal repeat sequence (ITR). An ITR sequence acts in cis to provide a functional origin of replication and allows for integration and excision of the vector from the genome of a cell. In preferred embodiments, one or more ITR sequences flank the nucleotide sequence encoding the melanopsin. The AAV genome typically also comprises packaging genes, such as rep and/or cap genes which encode packaging functions for an AAV particle. The rep gene encodes one or more of the proteins Rep78, Rep68, Rep52 and Rep40 or variants thereof. The cap gene encodes one or more capsid proteins such as VP1, VP2 and VP3 or variants thereof. These proteins make up the capsid of an AAV particle. Capsid variants are discussed below.

A promoter will be operably linked to each of the packaging genes. Specific examples of such promoters include the p5, p19 and p40 promoters (Laughlin et al. (1979) Proc. Natl. Acad. Sci. USA 76: 5567-5571). For example, the p5 and p19 promoters are generally used to express the rep gene, while the p40 promoter is generally used to express the cap gene.

As discussed above, the AAV genome used in the vector of the invention may therefore be the full genome of a naturally occurring AAV. For example, a vector comprising a full AAV genome may be used to prepare an AAV vector or vector particle in vitro. However, while such a vector may in principle be administered to patients, this will rarely be done in practice. Preferably the AAV genome will be derivatised for the purpose of administration to patients. Such derivatisation is standard in the art and the present invention encompasses the use of any known derivative of an AAV genome, and derivatives which could be generated by applying techniques known in the art. Derivatisation of the AAV genome and of the AAV capsid are reviewed in Coura and Nardi (2007) Virology Journal 4: 99, and in Choi et al. (2005) Curr. Gene Ther. 5: 299-310 and Wu et al. (2006) Molecular Therapy 14: 316-27.

Derivatives of an AAV genome include any truncated or modified forms of an AAV genome which allow for expression of a transgene from a vector of the invention in vivo. Typically, it is possible to truncate the AAV genome significantly to include minimal viral sequence yet retain the above function. This is preferred for safety reasons to reduce the risk of recombination of the vector with wild-type virus, and also to avoid triggering a cellular immune response by the presence of viral gene proteins in the target cell.

Typically, a derivative will include at least one inverted terminal repeat sequence (ITR), preferably more than one ITR, such as two ITRs or more. One or more of the ITRs may be derived from AAV genomes having different serotypes, or may be a chimeric or mutant ITR. A preferred mutant ITR is one having a deletion of a trs (terminal resolution site). This deletion allows for continued replication of the genome to generate a single-stranded genome which contains both coding and complementary sequences, i.e. a self-complementary AAV genome. This allows for bypass of DNA replication in the target cell, and so enables accelerated transgene expression.

The one or more ITRs will preferably flank the nucleotide sequence encoding the melanopsin at either end. The inclusion of one or more ITRs is preferred to aid concatamer formation of the vector of the invention in the nucleus of a host cell, for example following the conversion of single-stranded vector DNA into double-stranded DNA by the action of host cell DNA polymerases. The formation of such episomal concatamers protects the vector construct during the life of the host cell, thereby allowing for prolonged expression of the transgene in vivo.

In preferred embodiments, ITR elements will be the only sequences retained from the native AAV genome in the derivative. Thus, a derivative will preferably not include the rep and/or cap genes of the native genome and any other sequences of the native genome. This is preferred for the reasons described above, and also to reduce the possibility of integration of the vector into the host cell genome. Additionally, reducing the size of the AAV genome allows for increased flexibility in incorporating other sequence elements (such as regulatory elements) within the vector in addition to the transgene.

The following portions could therefore be removed in a derivative of the invention: one inverted terminal repeat (ITR) sequence, the replication (rep) and capsid (cap) genes. However, in some embodiments, derivatives may additionally include one or more rep and/or cap genes or other viral sequences of an AAV genome. Naturally occurring AAV integrates with a high frequency at a specific site on human chromosome 19, and shows a negligible frequency of random integration, such that retention of an integrative capacity in the vector may be tolerated in a therapeutic setting.

Where a derivative comprises capsid proteins i.e. VP1, VP2 and/or VP3, the derivative may be a chimeric, shuffled or capsid-modified derivative of one or more naturally occurring AAVs. In particular, the invention encompasses the provision of capsid protein sequences from different serotypes, clades, clones, or isolates of AAV within the same vector i.e. pseudotyping.

Chimeric, shuffled or capsid-modified derivatives will be typically selected to provide one or more desired functionalities for the viral vector. Thus, these derivatives may display increased efficiency of gene delivery, decreased immunogenicity (humoral or cellular), an altered tropism range and/or improved targeting of a particular cell type compared to an AAV vector comprising a naturally occurring AAV genome, such as that of AAV2. Increased efficiency of gene delivery may be effected by improved receptor or co-receptor binding at the cell surface, improved internalisation, improved trafficking within the cell and into the nucleus, improved uncoating of the viral particle and improved conversion of a single-stranded genome to double-stranded form. Increased efficiency may also relate to an altered tropism range or targeting of a specific cell population, such that the vector dose is not diluted by administration to tissues where it is not needed.

Chimeric capsid proteins include those generated by recombination between two or more capsid coding sequences of naturally occurring AAV serotypes. This may be performed for example by a marker rescue approach in which non-infectious capsid sequences of one serotype are co-transfected with capsid sequences of a different serotype, and directed selection is used to select for capsid sequences having desired properties. The capsid sequences of the different serotypes can be altered by homologous recombination within the cell to produce novel chimeric capsid proteins.

Chimeric capsid proteins also include those generated by engineering of capsid protein sequences to transfer specific capsid protein domains, surface loops or specific amino acid residues between two or more capsid proteins, for example between two or more capsid proteins of different serotypes.

Shuffled or chimeric cap sid proteins may also be generated by DNA shuffling or by error-prone PCR. Hybrid AAV capsid genes can be created by randomly fragmenting the sequences of related AAV genes e.g. those encoding capsid proteins of multiple different serotypes and then subsequently reassembling the fragments in a self-priming polymerase reaction, which may also cause crossovers in regions of sequence homology. A library of hybrid AAV genes created in this way by shuffling the capsid genes of several serotypes can be screened to identify viral clones having a desired functionality. Similarly, error prone PCR may be used to randomly mutate AAV cap sid genes to create a diverse library of variants which may then be selected for a desired property.

The sequences of the capsid genes may also be genetically modified to introduce specific deletions, substitutions or insertions with respect to the native wild-type sequence. In particular, capsid genes may be modified by the insertion of a sequence of an unrelated protein or peptide within an open reading frame of a capsid coding sequence, or at the N-and/or C-terminus of a capsid coding sequence.

The unrelated protein or peptide may advantageously be one which acts as a ligand for a particular cell type, thereby conferring improved binding to a target cell or improving the specificity of targeting of the vector to a particular cell population. An example might include the use of RGD peptide to block uptake in the retinal pigment epithelium and thereby enhance transduction of surrounding retinal tissues (Cronin et al. (2008) ARVO Abstract: D1048). The unrelated protein may also be one which assists purification of the viral particle as part of the production process, i.e. an epitope or affinity tag. The site of insertion will typically be selected so as not to interfere with other functions of the viral particle, e.g. internalisation or trafficking of the viral particle. The skilled person can identify suitable sites for insertion based on their common general knowledge. Particular sites are disclosed in Choi et al. (2005) Curr. Gene Ther. 5: 299-310.

The invention additionally encompasses the provision of sequences of an AAV genome in a different order and configuration to that of a native AAV genome. The invention also encompasses the replacement of one or more AAV sequences or genes with sequences from another virus or with chimeric genes composed of sequences from more than one virus. Such chimeric genes may be composed of sequences from two or more related viral proteins of different viral species.

The vector of the invention may take the form of a nucleotide sequence comprising an AAV genome or derivative thereof and a sequence encoding the melanopsin transgene or a variant thereof.

The AAV particles of the invention include transcapsidated forms wherein an AAV genome or derivative having an ITR of one serotype is packaged in the capsid of a different serotype. The AAV particles of the invention also include mosaic forms wherein a mixture of unmodified capsid proteins from two or more different serotypes makes up the viral capsid. The AAV particle also includes chemically modified forms bearing ligands adsorbed to the capsid surface. For example, such ligands may include antibodies for targeting a particular cell surface receptor.

Thus, for example, the AAV particles of the invention include those with an AAV2 genome and AAV2 capsid proteins (AAV2/2), those with an AAV2 genome and AAV5 cap sid proteins (AAV2/5) and those with an AAV2 genome and AAV8 capsid proteins (AAV2/8).

A preferred capsid protein for use in the present invention is the AAV8 Y733F mutant capsid (Petrs-Silva, H. et al. (2009) Mol. Ther. 17: 463-71).

Herein, amino acid residues within the AAV8 capsid are numbered following a convention whereby the N-terminal methionine of the example AAV8 capsid deposited under NCBI Accession No. YP_077180.1 (SEQ ID NO: 5) is assigned to be residue 1 (Y733 has been underlined in SEQ ID NO: 5, below).

(SEQ ID NO: 5)
MAADGYLPDWLEDNLSEGIREWWALKPGAPKPKANQQKQDDGRGLVLPGY
KYLGPFNGLDKGEPVNAADAAALEHDKAYDQQLQAGDNPYLRYNHADAEF
QERLQEDTSFGGNLGRAVFQAKKRVLEPLGLVEEGAKTAPGKKRPVEPSP
QRSPDSSTGIGKKGQQPARKRLNFGQTGDSESVPDPQPLGEPPAAPSGVG
PNTMAAGGGAPMADNNEGADGVGSSSGNWHCDSTWLGDRVITTSTRTWAL
PTYNNHLYKQISNGTSGGATNDNTYFGYSTPWGYFDFNRFHCHFSPRDWQ
RLINNNWGFRPKRLSFKLFNIQVKEVTQNEGTKTIANNLTSTIQVFTDSE
YQLPYVLGSAHQGCLPPFPADVFMIPQYGYLTLNNGSQAVGRSSFYCLEY
FPSQMLRTGNNFQFTYTFEDVPFHSSYAHSQSLDRLMNPLIDQYLYYLSR
TQTTGGTANTQTLGFSQGGPNTMANQAKNWLPGPCYRQQRVSTTTGQNNN
SNFAWTAGTKYHLNGRNSLANPGIAMATHKDDEERFFPSNGILIFGKQNA
ARDNADYSDVMLTSEEEIKTTNPVATEEYGIVADNLQQQNTAPQIGTVNS
QGALPGMVWQNRDVYLQGPIWAKIPHTDGNFHPSPLMGGFGLKHPPPQIL
IKNTPVPADPPTTFNQSKLNSFITQYSTGQVSVEIEWELQKENSKRWNPE
IQYTSNYYKSTSVDFAVNTEGVYSEPRPIGTRYLTRNL

Identities of individual residues and positions of mutations are described herein with reference to this numbering convention. A skilled person would readily be able to determine analogous positions in homologous proteins by performing a sequence alignment to this capsid protein.

### Promoters and Regulatory Sequences

The vector of the invention may also include elements allowing for the expression of the melanopsin transgene in vitro or in vivo. These may be referred to as expression control sequences. Thus, the vector typically comprises expression control sequences (e.g. comprising a promoter sequence) operably linked to the nucleotide sequence encoding the transgene.

By “operably linked”, it is to be understood that the individual components are linked together in a manner which enables them to carry out their function substantially unhindered (e.g. a promoter may be operably linked to a nucleotide of interest to promote expression of the nucleotide of interest in a cell).

Any suitable promoter may be used, the selection of which may be readily made by the skilled person. The promoter sequence may be constitutively active (i.e. operational in any host cell background), or alternatively may be active only in a specific host cell environment, thus allowing for targeted expression of the transgene in a particular cell type (e.g. a tissue-specific promoter). The promoter may show inducible expression in response to presence of another factor, for example a factor present in a host cell. In any event, where the vector is administered for therapy, it is preferred that the promoter should be functional in the target cell background.

In some embodiments, it is preferred that the promoter shows retinal-cell specific expression in order to allow for the transgene to only be expressed in retinal cell populations. Thus, expression from the promoter may be retinal-cell specific, for example confined only to cells of the neurosensory retina and retinal pigment epithelium.

Preferred promoters include the chicken beta-actin (CBA) promoter, optionally in combination with a cytomegalovirus (CMV) enhancer element. An example promoter for use in the invention is a hybrid CBA/CAG promoter, for example the promoter used in previous clinical trials (MacLaren, R. E. et al. (2014) Lancet 383: 1129-37). A further example promoter for use in the invention has the sequence:

(SEQ ID NO: 6)
ATTGACGTCAATAATGACGTATGTTCCCATAGTAACGCCAATAGGGACTT
TCCATTGACGTCAATGGGTGGAGTATTTACGGTAAACTGCCCACTTGGCA
GTACATCAAGTGTATCATATGCCAAGTACGCCCCCTATTGACGTCAATGA
CGGTAAATGGCCCGCCTGGCATTATGCCCAGTACATGACCTTATGGGACT
TTCCTACTTGGCAGTACATCTACGTATTAGTCATCGCTATTACCATGGTC
GAGGTGAGCCCCACGTTCTGCTTCACTCTCCCCATCTCCCCCCCCTCCCC
ACCCCCAATTTTGTATTTATTTATTTTTTAATTATTTTGTGCAGCGATGG
GGGCGGGGGGGGGGGGGGGGCGCGCGCCAGGCGGGGCGGGGCGGGGCGAG
GGGCGGGGCGGGGCGAGGCGGAGAGGTGCGGCGGCAGCCAATCAGAGCGG
CGCGCTCCGAAAGTTTCCTTTTATGGCGAGGCGGCGGCGGCGGCGGCCCT
ATAAAAAGCGAAGCGCGCGGCGGGCGGGAGTCGCTGCGCGCTGCCTTCGC
CCCGTGCCCCGCTCCGCCGCCGCCTCGCGCCGCCCGCCCCGGCTCTGACT
GACCGCGTTACTCCCACAGGTGAGCGGGCGGGACGGCCCTTCTCCTCCGG
GCTGTAATTAGCGCTTGGTTTAATGACGGCTTGTTTCTTTTCTGTGGCTG
CGTGAAAGCCTTGAGGGGCTCCGGGAGGGCCCTTTGTGCGGGGGGAGCGG
CTCGGGGCTGTCCGCGGGGGGACGGCTGCCTTCGGGGGGGACGGGGCAGG
GCGGGGTTCGGCTTCTGGCGTGTGACCGGCGGCTCTAGAGCCTCTGCTAA
CCATGTTCATGCCTTCTTCTTTTTCCTACAGCTCCTGGGCAACGTGCTGG
TTATTGTGCTGTCTCATCATTTTGGCAAAGAATT

The vector of the invention may also comprise one or more additional regulatory sequences with may act pre- or post-transcriptionally. The regulatory sequence may be part of the native transgene locus or may be a heterologous regulatory sequence. The vector of the invention may comprise portions of the 5′-UTR or 3′-UTR from the native transgene transcript.

Regulatory sequences are any sequences which facilitate expression of the transgene, i.e. act to increase expression of a transcript, improve nuclear export of mRNA or enhance its stability. Such regulatory sequences include for example enhancer elements, postregulatory elements and polyadenylation sites. A preferred polyadenylation site is the Bovine Growth Hormone poly-A signal which may be as shown below:

(SEQ ID NO: 7)
TCGCTGATCAGCCTCGACTGTGCCTTCTAGTTGCCAGCCATCTGTTGTTT
GCCCCTCCCCCGTGCCTTCCTTGACCCTGGAAGGTGCCACTCCCACTGTC
CTTTCCTAATAAAATGAGGAAATTGCATCGCATTGTCTGAGTAGGTGTCA
TTCTATTCTGGGGGGTGGGGTGGGGCAGGACAGCAAGGGGGAGGATTGGG
AAGACAATAGCAGGCATGCTGGGGATGCGGTGGGCTCTATGGCTTCTGAG
GCGGAAAGAACCAGCTGGGG

In the context of the vector of the invention, such regulatory sequences will be cis-acting. However, the invention also encompasses the use of trans-acting regulatory sequences located on additional genetic constructs.

A preferred post-regulatory element for use in a vector of the invention is the woodchuck hepatitis postregulatory element (WPRE) or a variant thereof. An example sequence of the WPRE is shown below:

(SEQ ID NO: 8)
ATCAACCTCTGGATTACAAAATTTGTGAAAGATTGACTGGTATTCTTAAC
TATGTTGCTCCTTTTACGCTATGTGGATACGCTGCTTTAATGCCTTTGTA
TCATGCTATTGCTTCCCGTATGGCTTTCATTTTCTCCTCCTTGTATAAAT
CCTGGTTGCTGTCTCTTTATGAGGAGTTGTGGCCCGTTGTCAGGCAACGT
GGCGTGGTGTGCACTGTGTTTGCTGACGCAACCCCCACTGGTTGGGGCAT
TGCCACCACCTGTCAGCTCCTTTCCGGGACTTTCGCTTTCCCCCTCCCTA
TTGCCACGGCGGAACTCATCGCCGCCTGCCTTGCCCGCTGCTGGACAGGG
GCTCGGCTGTTGGGCACTGACAATTCCGTGGTGTTGTCGGGGAAATCATC
GTCCTTTCCTTGGCTGCTCGCCTGTGTTGCCACCTGGATTCTGCGCGGGA
CGTCCTTCTGCTACGTCCCTTCGGCCCTCAATCCAGCGGACCTTCCTTCC
CGCGGCCTGCTGCCGGCTCTGCGGCCTCTTCCGCGTCTTCGCCTTCGCCC
TCAGACGAGTCGGATCTCCCTTTGGGCCGCCTCCCCGC

The invention encompasses the use of any valiant sequence of the WPRE which increases expression of the transgene compared to a vector without a WPRE. Preferably, variant sequences display at least 70% homology to SEQ ID NO: 8 over its entire sequence, more preferably 75%, 80%, 85%, 90% and more preferably at least 95%, 96% 97%, 98% or 99% homology to SEQ ID NO: 8 over its entire sequence.

Another regulatory sequence which may be used in a vector of the present invention is a scaffold-attachment region (SAR). Additional regulatory sequences may be readily selected by the skilled person.

### Method of Administration

The AAV vector of the present invention is administered to the eye of a subject by subretinal injection.

The skilled person will be familiar with and well able to carry out individual subretinal injections.

### Subretinal Injection

Subretinal injections are injections into the subretinal space, i.e. underneath the neurosensory retina. During a subretinal injection, the injected material is directed into, and creates a space between, the photoreceptor cell and retinal pigment epithelial (RPE) layers.

When the injection is carried out through a small retinotomy, a retinal detachment may be created. The detached, raised layer of the retina that is generated by the injected material is referred to as a “bleb”.

The hole created by the subretinal injection must be sufficiently small that the injected solution does not significantly reflux back into the vitreous cavity after administration. Such reflux would be particularly problematic when a medicament is injected, because the effects of the medicament would be directed away from the target zone. Preferably, the injection creates a self-sealing entry point in the neurosensory retina, i.e. once the injection needle is removed, the hole created by the needle reseals such that very little or substantially no injected material is released through the hole.

To facilitate this process, specialist subretinal injection needles are commercially available (e.g. DORC 41G Teflon subretinal injection needle, Dutch Ophthalmic Research Center International BY, Zuidland, The Netherlands). These are needles designed to carry out subretinal injections.

Unless damage to the retina occurs during the injection, and as long as a sufficiently small needle is used, substantially all injected material remains localised between the detached neurosensory retina and the RPE at the site of the localised retinal detachment (i.e. does not reflux into the vitreous cavity). Indeed, the typical persistence of the bleb over a short time frame indicates that there is usually little escape of the injected material into the vitreous. The bleb may dissipate over a longer time frame as the injected material is absorbed.

Visualisations of the eye, in particular the retina, for example using optical coherence tomography, may be made pre-operatively.

### Two-Step Subretinal Injection

The vector of the present invention may be delivered with increased accuracy and safety by using a two-step method in which a localised retinal detachment is created by the subretinal injection of a first solution. The first solution does not comprise the vector. A second subretinal injection is then used to deliver the medicament comprising the vector into the subretinal fluid of the bleb created by the first subretinal injection. Because the injection delivering the medicament is not being used to detach the retina, a specific volume of solution may be injected in this second step.

In one embodiment of the present invention, the AAV vector is delivered by:
(a) administering a solution to the subject by subretinal injection in an amount effective to at least partially detach the retina to form a subretinal bleb, wherein the solution does not comprise the AAV vector; and
(b) administering a medicament composition by subretinal injection into the bleb formed by step (a), wherein the medicament comprises the AAV vector.

The volume of solution injected in step (a) to at least partially detach the retina may be, for example, about 10-1000 μL, for example about 50-1000, 100-1000, 250-1000, 500-1000, 10-500, 50-500, 100-500, 250-500 μL. The volume may be, for example, about 10, 50, 100, 200, 300, 400, 500, 600, 700, 800, 900 or 1000 μL.

The volume of the medicament composition injected in step (b) may be, for example, about 10-500 μL, for example about 50-500, 100-500, 200-500, 300-500, 400-500, 50-250, 100-250, 200-250 or 50-150 μL. The volume may be, for example, about 10, 50, 100, 150, 200, 250, 300, 350, 400, 450 or 500 μL. Preferably, the volume of the medicament composition injected in step (b) is 100 μL. Larger volumes may increase the risk of stretching the retina, while smaller volumes may be difficult to see.

The solution that does not comprise the medicament (i.e. the “first solution” of step (a)) may be similarly formulated to the solution that does comprise the medicament, as described below. A preferred solution that does not comprise the medicament is balanced saline solution (BSS) or a similar buffer solution matched to the pH and osmolality of the subretinal space.

### Visualising the Retina During Surgery

Under certain circumstances, for example during end-stage retinal degenerations, identifying the retina is difficult because it is thin, transparent and difficult to see against the disrupted and heavily pigmented epithelium on which it sits. The use of a blue vital dye (e.g. Brilliant Peel®, Geuder; MembraneBlue-Dual®, Dorc) may facilitate the identification of the retinal hole made for the retinal detachment procedure (i.e. step (a) in the two-step subretinal injection method of the present invention) so that the medicament can be administered through the same hole without the risk of reflux back into the vitreous cavity.

The use of the blue vital dye also identifies any regions of the retina where there is a thickened internal limiting membrane or epiretinal membrane, as injection through either of these structures would hinder clean access into the subretinal space. Furthermore, contraction of either of these structures in the immediate post-operative period could lead to stretching of the retinal entry hole, which could lead to reflux of the medicament into the vitreous cavity.

### Pharmaceutical Compositions and Injected Solutions

The medicaments, for example vectors, of the present invention may be formulated into pharmaceutical compositions. These compositions may comprise, in addition to the medicament, a pharmaceutically acceptable carrier, diluent, excipient, buffer, stabiliser or other materials well known in the art. Such materials should be non-toxic and should not interfere with the efficacy of the active ingredient. The precise nature of the carrier or other material may be determined by the skilled person according to the route of administration, i.e. subretinal injection.

The pharmaceutical composition is typically in liquid form. Liquid pharmaceutical compositions generally include a liquid carrier such as water, petroleum, animal or vegetable oils, mineral oil or synthetic oil. Physiological saline solution, magnesium chloride, dextrose or other saccharide solution, or glycols such as ethylene glycol, propylene glycol or polyethylene glycol may be included. In some cases, a surfactant, such as pluronic acid (PF68) 0.001% may be used.

For injection at the site of affliction, the active ingredient may be in the form of an aqueous solution which is pyrogen-free, and has suitable pH, isotonicity and stability. The skilled person is well able to prepare suitable solutions using, for example, isotonic vehicles such as Sodium Chloride Injection, Ringer's Injection or Lactated Ringer's Injection. Preservatives, stabilisers, buffers, antioxidants and/or other additives may be included as required.

For delayed release, the medicament may be included in a pharmaceutical composition which is formulated for slow release, such as in microcapsules formed from biocompatible polymers or in liposomal carrier systems according to methods known in the art.

### Method of Treatment

It is to be appreciated that all references herein to treatment include curative, palliative and prophylactic treatment; although in the context of the present invention references to preventing are more commonly associated with prophylactic treatment. Treatment may also include arresting progression in the severity of a disease.

The treatment of mammals, particularly humans, is preferred. However, both human and veterinary treatments are within the scope of the present invention.

### Variants, Derivatives, Analogues, Homologues and Fragments

In addition to the specific proteins and nucleotides mentioned herein, the present invention also encompasses the use of variants, derivatives, analogues, homologues and fragments thereof.

In the context of the present invention, a variant of any given sequence is a sequence in which the specific sequence of residues (whether amino acid or nucleic acid residues) has been modified in such a manner that the polypeptide or polynucleotide in question substantially retains its function. A variant sequence can be obtained by addition, deletion, substitution, modification, replacement and/or variation of at least one residue present in the naturally-occurring protein or polynucleotide.

The term “derivative” as used herein, in relation to proteins or polypeptides of the present invention includes any substitution of, variation of, modification of, replacement of, deletion of and/or addition of one (or more) amino acid residues from or to the sequence, providing that the resultant protein or polypeptide substantially retains at least one of its endogenous functions.

The term “analogue” as used herein, in relation to polypeptides or polynucleotides includes any mimetic, that is, a chemical compound that possesses at least one of the endogenous functions of the polypeptides or polynucleotides which it mimics.

Typically, amino acid substitutions may be made, for example from 1, 2 or 3 to 10 or 20 substitutions, provided that the modified sequence substantially retains the required activity or ability. Amino acid substitutions may include the use of non-naturally occurring analogues.

Proteins used in the present invention may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity and/or the amphipathic nature of the residues as long as the endogenous function is retained. For example, negatively charged amino acids include aspartic acid and glutamic acid; positively charged amino acids include lysine and arginine; and amino acids with uncharged polar head groups having similar hydrophilicity values include asparagine, glutamine, serine, threonine and tyrosine.

Conservative substitutions may be made, for example according to the table below. Amino acids in the same block in the second column and preferably in the same line in the third column may be substituted for each other:

| | | |
|---|---|---|
| ALIPHATIC | Non-polar | G A P |
| | | I L V |
| | Polar-uncharged | C S T M |
| | | N Q |
| | Polar-charged | D E |
| | | K R H |
| AROMATIC | | F W Y |

The term “homologue” as used herein means an entity having a certain homology with the wild type amino acid sequence and the wild type nucleotide sequence. The term “homology” can be equated with “identity”.

A homologous sequence may include an amino acid sequence which may be at least 50%, 55%, 60%, 65%, 70%, 75%, 80%, 85% or 90% identical, preferably at least 95%, 96%, 97%, 98% or 99% identical to the subject sequence. Typically, the homologues will comprise the same active sites etc. as the subject amino acid sequence. Although homology can also be considered in terms of similarity (i.e. amino acid residues having similar chemical properties/functions), in the context of the present invention it is preferred to express homology in terms of sequence identity.

A homologous sequence may include a nucleotide sequence which may be at least 50%, 55%, 60%, 65%, 70%, 75%, 80%, 85% or 90% identical, preferably at least 95%, 96%, 97%, 98% or 99% identical to the subject sequence. Although homology can also be considered in terms of similarity, in the context of the present invention it is preferred to express homology in terms of sequence identity.

Preferably, reference to a sequence which has a percent identity to any one of the SEQ ID NOs detailed herein refers to a sequence which has the stated percent identity over the entire length of the SEQ ID NO referred to.

Homology comparisons can be conducted by eye or, more usually, with the aid of readily available sequence comparison programs. These commercially available computer programs can calculate percent homology or identity between two or more sequences.

Percent homology may be calculated over contiguous sequences, i.e. one sequence is aligned with the other sequence and each residue in one sequence is directly compared with the corresponding residue in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion in the amino acid or nucleotide sequence may cause the following residues or codons to be put out of alignment, thus potentially resulting in a large reduction in percent homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical residues, a sequence alignment with as few gaps as possible, reflecting higher relatedness between the two compared sequences, will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example, when using the GCG Wisconsin Bestfit package the default gap penalty for amino acid sequences is −12 for a gap and −4 for each extension.

Calculation of maximum percent homology therefore firstly requires the production of an optimal alignment, taking into consideration gap penalties. A suitable computer program for carrying out such an alignment is the GCG Wisconsin Bestfit package (University of Wisconsin, USA.; Devereux et al. (1984) Nucleic Acids Res. 12: 387). Examples of other software that can perform sequence comparisons include, but are not limited to, the BLAST package (see Ausubel et al. (1999) ibid—Ch. 18), FASTA (Atschul et al. (1990) J. Mol. Biol. 403-410) and the GENEWORKS suite of comparison tools. Both BLAST and PASTA are available for offline and online searching (see Ausubel et al. (1999) ibid, pages 7-58 to 7-60). However, for some applications, it is preferred to use the GCG Bestfit program. Another tool, called BLAST 2 Sequences is also available for comparing protein and nucleotide sequences (see ELMS Microbiol. Lett. (1999) 174: 247-50; FEMS Microbiol. Lett. (1999) 177: 187-8).

Although the final percent homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see the user manual for further details). For some applications, it is preferred to use the public default values for the GCG package, or in the case of other software, the default matrix, such as BLOSUM62.

Once the software has produced an optimal alignment, it is possible to calculate percent homology, preferably percent sequence identity. The software typically does this as part of the sequence comparison and generates a numerical result.

“Fragments” of full length melanopsin are also variants and the term typically refers to a selected region of the polypeptide or polynucleotide that is of interest either functionally or, for example, in an assay. “Fragment” thus refers to an amino acid or nucleic acid sequence that is a portion of a full-length polypeptide or polynucleotide.

Such variants may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site may be made. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded protein. These methods are only illustrative of the numerous standard techniques known in the art for manipulation of DNA sequences and other known techniques may also be used.

### Codon Optimisation

The polynucleotides used in the present invention may be codon-optimised. Codon optimisation has previously been described in WO 1999/41397 and WO 2001/79518. Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available.

### EXAMPLES

### Example 1

The present inventors targeted the bipolar cells and horizontal cells of the retina, which are next in line after photoreceptor degeneration, for the introduction of exogenous melanopsin. To achieve this, the inventors injected an AAV vector under the retina (i.e. by subretinal injection) in retinal degeneration (rd) mice, which have the human equivalent of end-stage RP (FIGS. 1A-1D). The treated mice developed significantly increased pupil responses to light (FIGS. 2A-2E) and the location of invoked electrical recordings correlated to the regions where bipolar and horizontal cells had been transduced (FIGS. 2A-2E).

One of the advantages of targeting the bipolar and horizontal cells is that they more closely represent the map that would previously have been formed by the photo-receptors. Although ganglion cells could be made light-sensitive, the axons of the ganglion cells run horizontally across the retina to the optic nerve head and it is difficult to predict how vision would be useful without some form of electronic interface to reprogram the nerve signals.

Furthermore, gene therapy using intravitreal injections may be more prone to inflammation as the vector more easily escapes from the eye. It would also be possible to apply higher concentrations of the vector using the subretinal approach.

Targeting the bipolar and horizontal cells has the additional advantage of feeding the light-sensitive signals in at the most upstream part of the normal visual pathway.

In addition, the present inventors have improved the transduction of the retina using a novel type of AAV vector. The AAV vector has a mutation in one of the tyrosine residues in the capsid protein (AAV8 Y733F capsid mutant). This makes the vector considerably more efficient at expressing its gene product in cells of the retina.

This study used an AAV vector harbouring a melanopsin gene sequence. However, a gene for a red fluorescent protein (DsRed) was also incorporated into the vector, in order to study which cells were infected by the vector (FIG. 1A-1D and FIG. 2A-2E). This also confirmed that the vector was still efficacious 15 months after the initial subretinal injection.

### Example 2

Histology confirmed the transduction of the outer retinal cells (bipolar and horizontal) that do not normally express melanopsin (FIGS. 1A-1D).

Widespread transduction of the retina of the rdl mouse (which has a rapid photoreceptor degeneration) was observed by immune-staining for human melanopsin (FIGS. 1A-1B—4 months (FIG. 1A left panel a i) and 15 months (FIG. 1B left panel b i) after gene therapy; images of control retinae are shown for comparison showing absence of human melanopsin at 4 months (FIG. 1A right panel iii) and 15 months (FIG. 1B right panel iii)).

The red label (DsRed) in the top right panel of FIG. 1C identifies transduced bipolar and horizontal cells on the subretinal surf ace. Appropriate membrane localisation of human melanopsin was also observed, demonstrating the network of transduced cells.

Further immunostaining for PKC alpha (identifying bipolar cells) and calbindin (identifying horizontal cells) illustrated transduction of these cell types in the degenerate retina using the melanopsin vector (FIG. 1D, bottom series of six panels).

### Example 3

### Methods

Capsid mutant AAV (rAAV2/8.Y733F) expressing human melanopsin was delivered via subretinal injection to the left eye of 15 Pde6brd1/rd1 mice. 9 mice were injected with phosphate-buffered saline (sham group) and 13 untreated mice were also included. 12 months after treatment, pupil responses were recorded from the right eye following stimulation of the left eye with a 2 second white light stimulus of varying intensity.

### Results

12 months after subretinal injection, a large area of retina in each treated eye expressed human melanopsin, as confirmed by immunohistochemistry. At a light intensity of 2×10¹⁵ photons/cm²/s, which was in the mid-range of the light intensities tested, there was significantly more pupil constriction in the treated group (mean constriction 36.0% ± 7.06) versus the sham treated group (3.1% ± 1.45) and the untreated group (15.6% ± 6.61); treated versus sham p=0.0026, treated versus untreated p=0.038 2 way ANOVA, Tukey's post hoc test.

### Conclusions

Human melanopsin can be successfully delivered via subretinal injection to the mouse degenerate retina using capsid mutant AA V, with a sustained high level of gene expression seen after 12 months. The pupil light response in treated animals was significantly improved compared to sham-injected and untreated mice, providing evidence that human melanopsin mediated photosensitivity can restore visual function in the degenerate retina.

### Example 4

The present inventors also studied visually evoked readings from the cortex of blind rdl mice after transduction with an AAV vector harbouring the melanopsin gene. The readings showed changes in blood flow, a biomarker of increased neuronal activity, in a part of the brain that is not a target of the melanopsin expressing ganglion cells normally present in the eye. This study provides additional proof of activation of a novel pathway.

Further experiments carried out by the present inventors related to object recognition (another functional test) and showed a statistically significant improvement following administration of the AAV vector. Object recognition would most likely require cortical activation.

The technique of laser speckle cortical imaging was used to assess blood flow changes in the visual cortex following a 2 second 480 nm light stimulus. A photograph of exposed skull is shown in FIG. 3A which indicates orientation and the location of the visual cortex. Activation in response to visual stimulation in a treated mouse is overlaid, with a large response corresponding to the right visual cortex, and a small response corresponding to the left visual cortex. Blood flow changes in the visual cortex were measured in treated (n=6) and sham-injected (n=5) mice. Mean responses in both groups are shown in FIG. 3B.

Visual environment recognition tests were also carried out in mice treated with human melanopsin compared to controls at 12 months after injection. Schematic FIG. 3C illustrates tests where each animal has two trials where the visual environment is constant (same); or where there is a change in visual environment during the task from a black and white patterned box to a plain white box (change). Measure of behaviour (FIG. 3D) with and without change in environment for each group demonstrated a significant difference in behaviour in wild type and treated mice when the visual environment is changed (** p<0.01, 2 way ANOVA with Tukey post hoc test).

All publications mentioned in the above specification are herein incorporated by reference. Various modifications and variations of the described methods of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention, which are obvious to those skilled in biochemistry and biotechnology or related fields, are intended to be within the scope of the following claims.

1. A method of treating an eye disease comprising administering an adeno-associated virus (AAV) vector to a mammalian subject by subretinal injection, wherein the AAV vector comprises a nucleotide sequence encoding melanopsin operably linked to an expression control sequence to promote expression of melanopsin in cells of the eye of the subject.

2. The method of claim 1, wherein the eye disease is a retinal dystrophy.

3. The method of claim 1, wherein the eye disease is retinitis pigmentosa.

4. The method of claim 1, wherein the eye to be treated lacks rod and/or cone photoreceptor cells.

5. The method of claim 1, wherein the AAV vector is in the form of an AAV particle comprising an AAV8 Y733F mutant capsid.

6. The method of claim 1, wherein the AAV vector comprises an AAV2 genome.

7. The method of claim 1, wherein the melanopsin is expressed in bipolar and/or horizontal cells.

8. The method of claim 1, wherein the melanopsin is human melanopsin.

9. The method of claim 1, wherein the expression control sequence comprises a CBA promoter.

10. The method of claim 1, wherein the subretinal injection comprises the steps:
(a) administering a solution to the subject by subretinal injection in an amount effective to at least partially detach the retina to form a subretinal bleb, wherein the solution does not comprise the AAV vector; and
(b) administering a medicament composition by subretinal injection into the bleb formed by step (a), wherein the medicament comprises the AAV vector.

11. A method of improving or restoring vision comprising administering an adeno-associated virus (AAV) vector to a mammalian subject by subretinal injection, wherein the AAV vector comprises a nucleotide sequence encoding melanopsin operably linked to an expression control sequence to promote expression of melanopsin in cells of the eye of the subject.

12. The method of claim 11, wherein the subject suffers from a retinal dystrophy.

13. The method of claim 11, wherein the subject suffers from retinitis pigmentosa.

14. The method of claim 11, wherein the eye to be treated lacks rod and/or cone photoreceptor cells.

15. The method of claim 11, wherein the AAV vector is in the form of an AAV particle comprising an AAV8 Y733F mutant capsid.

16. The method of claim 11, wherein the AAV vector comprises an AAV2 genome.

17. The method of claim 11, wherein the melanopsin is expressed in bipolar and/or horizontal cells.

18. The method of claim 11, wherein the melanopsin is human melanopsin.

19. The method of claim 11, wherein the expression control sequence comprises a CBA promoter.

20. The method of claim 11, wherein the subretinal injection comprises the steps:
(a) administering a solution to the subject by subretinal injection in an amount effective to at least partially detach the retina to form a subretinal bleb, wherein the solution does not comprise the AAV vector; and
(b) administering a medicament composition by subretinal injection into the bleb formed by step (a), wherein the medicament comprises the AAV vector.

21. The method of claim 1, wherein the eye to be treated has less than 50% of the functional rod and/or cone photoreceptor cells that were present prior to the onset of the disease.

22. The method of claim 11, wherein the eye to be treated has less than 50% of the functional rod and/or cone photoreceptor cells that were present prior to the onset of the disease.


SEQUENCE LISTING (8 sequences)

SEQ ID NO: 1 - DNA, 1437 bp, Homo sapiens
atgaaccctccttcggggccaagagtcccgcccagcccaacccaagagcccagctgcatggccaccccagcaccacccagctggtgggacagctcccagagcagcatctccagcctgggccggcttccatccatcagtcccacagcacctgggacttgggctgctgcctgggtccccctccccacggttgatgttccagaccatgcccactataccctgggcacagtgatcttgctggtgggactcacggggatgctgggcaacctgacggtcatctataccttctgcaggagcagaagcctccggacacctgccaacatgttcattatcaacctcgcggtcagcgacttcctcatgtccttcacccaggcccctgtcttcttcaccagtagcctctataagcagtggctctttggggagacaggctgcgagttctatgccttctgtggagctctctttggcatttcctccatgatcaccctgacggccatcgccctggaccgctacctggtaatcacacgcccgctggccacctttggtgtggcgtccaagaggcgtgcggcatttgtcctgctgggcgtttggctctatgccctggcctggagtctgccacccttcttcggctggagcgcctacgtgcccgaggggttgctgacatcctgctcctgggactacatgagcttcacgccggccgtgcgtgcctacaccatgcttctctgctgcttcgtgttcttcctccctctgcttatcatcatctactgctacatcttcatcttcagggccatccgggagacaggacgggctctccagaccttcggggcctgcaagggcaatggcgagtccctgtggcagcggcagcggctgcagagcgagtgcaagatggccaagatcatgctgctggtcatcctcctcttcgtgctctcctgggctccctattccgctgtggccctggtggcctttgctgggtacgcacacgtcctgacaccctacatgagctcggtgccagccgtcatcgccaaggcctctgcaatccacaaccccatcatttacgccatcacccaccccaagtacagggtggccattgcccagcacctgccctgcctgggggtgctgctgggtgtatcacgccggcacagtcgcccctaccccagctaccgctccacccaccgctccacgctgaccagccacacctccaacctcagctggatctccatacggaggcgccaggagtccctgggctcggagagtgaggtgggctggacacacatggaggcagcagctgtgtggggagctgcccagcaagcaaatgggcggtccctctacggtcagggtctggaggacttggaagccaaggcaccccccagaccccagggacacgaagcagagactccagggaagaccaaggggctgatccccagccaggaccccaggatgtag

SEQ ID NO: 2 - DNA, 1470 bp, Homo sapiens
atgaaccctccttcggggccaagagtcccgcccagcccaacccaagagcccagctgcatggccaccccagcaccacccagctggtgggacagctcccagagcagcatctccagcctgggccggcttccatccatcagtcccacagcacctgggacttgggctgctgcctgggtccccctccccacggttgatgttccagaccatgcccactataccctgggcacagtgatcttgctggtgggactcacggggatgctgggcaacctgacggtcatctataccttctgcagagctgtgcttcgtggagtcactgtgatgatgcagagcagaagcctccggacacctgccaacatgttcattatcaacctcgcggtcagcgacttcctcatgtccttcacccaggcccctgtcttcttcaccagtagcctctataagcagtggctctttggggagacaggctgcgagttctatgccttctgtggagctctctttggcatttcctccatgatcaccctgacggccatcgccctggaccgctacctggtaatcacacgcccgctggccacctttggtgtggcgtccaagaggcgtgcggcatttgtcctgctgggcgtttggctctatgccctggcctggagtctgccacccttcttcggctggagcgcctacgtgcccgaggggttgctgacatcctgctcctgggactacatgagcttcacgccggccgtgcgtgcctacaccatgcttctctgctgcttcgtgttcttcctccctctgcttatcatcatctactgctacatcttcatcttcagggccatccgggagacaggacgggctctccagaccttcggggcctgcaagggcaatggcgagtccctgtggcagcggcagcggctgcagagcgagtgcaagatggccaagatcatgctgctggtcatcctcctcttcgtgctctcctgggctccctattccgctgtggccctggtggcctttgctgggtacgcacacgtcctgacaccctacatgagctcggtgccagccgtcatcgccaaggcctctgcaatccacaaccccatcatttacgccatcacccaccccaagtacagggtggccattgcccagcacctgccctgcctgggggtgctgctgggtgtatcacgccggcacagtcgcccctaccccagctaccgctccacccaccgctccacgctgaccagccacacctccaacctcagctggatctccatacggaggcgccaggagtccctgggctcggagagtgaggtgggctggacacacatggaggcagcagctgtgtggggagctgcccagcaagcaaatgggcggtccctctacggtcagggtctggaggacttggaagccaaggcaccccccagaccccagggacacgaagcagagactccagggaagaccaaggggctgatccccagccaggaccccaggatgtag

SEQ ID NO: 3 - protein, 478 aa, Homo sapiens
MNPPSGPRVPPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFTSSLYKQWLFGETGCEFYAFCGALFGISSMITLTAIALDRYLVITRPLATFGVASKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPKYRVAIAQHLPCLGVLLGVSRRHSRPYPSYRSTHRSTLTSHTSNLSWISIRRRQESLGSESEVGWTHMEAAAVWGAAQQANGRSLYGQGLEDLEAKAPPRPQGHEAETPGKTKGLIPSQDPRM

SEQ ID NO: 4 - protein, 489 aa, Homo sapiens
MNPPSGPRVPPSPTQEPSCMATPAPPSWWDSSQSSISSLGRLPSISPTAPGTWAAAWVPLPTVDVPDHAHYTLGTVILLVGLTGMLGNLTVIYTFCRAVLRGVTVMMQSRSLRTPANMFIINLAVSDFLMSFTQAPVFFTSSLYKQWLFGETGCEFYAFCGALFGISSMITLTAIALDRYLVITRPLATFGVASKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPKYRVAIAQHLPCLGVLLGVSRRHSRPYPSYRSTHRSTLTSHTSNLSWISIRRRQESLGSESEVGWTHMEAAAVWGAAQQANGRSLYGQGLEDLEAKAPPRPQGHEAETPGKTKGLIPSQDPRM

SEQ ID NO: 5 - protein, 738 aa, Adeno-associated virus
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 6 - DNA, 934 bp, Artificial Sequence
  Promotor sequence
attgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaatt

SEQ ID NO: 7 - DNA, 270 bp, Bos taurus
tcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctgggg

SEQ ID NO: 8 - DNA, 588 bp, Woodchuck hepatitis virus
atcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc